(19) United States
(12) Patent Application Publication
(10) Pub. No.: US 2014/0106388 A1
(43) Pub. Date: Apr. 17, 2014

(54) AUTOMATED CTC DETECTION

(71) Applicants: Joachim Bangert, Erlangen (DE); Katja Friedrich, Erlenbach a. Main (DE); Walter Gumbrecht, Herzogenaurach (DE); Karsten Hiltawsky, Schwerte (DE); Peter Paulicka, Roettenbach (DE); Manfred Stanzel, Berching (DE)

(72) Inventors: Joachim Bangert, Erlangen (DE); Katja Friedrich, Erlenbach a. Main (DE); Walter Gumbrecht, Herzogenaurach (DE); Karsten Hiltawsky, Schwerte (DE); Peter Paulicka, Roettenbach (DE); Manfred Stanzel, Berching (DE)

(73) Assignee: SIEMENS AKTIENGESELLSCHAFT, Munich (DE)

(21) Appl. No.: 14/118,701

(22) PCT Filed: Apr. 13, 2012
(86) PCT No.: PCT/EP2012/056802

§ 371 Date: Dec. 2, 2013

(30) Foreign Application Priority Data
May 20, 2011 (DE) 102011076221.3

(51) Int. Cl. G01N 33/50

(52) U.S. Cl. CPC G01N 33/5091
USPC 435/26; 435/34; 435/308.1

(57) ABSTRACT

An embodiment relates to a method and to an array for detecting live circulating or disseminated cells in body fluids (for example blood, urine) or tissue samples (for example bone marrow) mixed with liquid. The method includes: a) filtering the liquid sample through a porous membrane that is suitable for retaining cells to be detected, such that cells to be detected come to rest upon at least a part of the surface of the membrane and the sample liquid passes the membrane; b) applying a first process liquid containing a first agent for marking the cells to be detected with a first marker; c) incubating the process liquid on the membrane for a predetermined time period, wherein the cells to be detected are marked; and d) detecting the marked cells to be detected on the surface of the membrane.

### PRIORITY STATEMENT

This application is the national phase under 35 U.S.C. §371 of PCT International Application No. PCT/EP2012/056802 which has an International filing date of Apr. 13, 2012, which designated the United States of America and which claims priority to German patent application number DE 10 2011 076 221.3 filed May 20, 2011, the entire contents of each of which are hereby incorporated herein by reference.

### Field

At least one embodiment of the invention is in the field of in vitro diagnostics and generally relates to a method and/or to an array for detecting live, circulating or disseminated cells from body fluids (e.g. blood, urine) or tissue samples (e.g. bone marrow) mixed with liquid. The method according to an embodiment of the invention serves in particular for the automated isolation and analysis of circulating tumor cells and is thus preferably used in tumor diagnostics.

At least one embodiment of the invention permits the automated detection of cells or cell constituents from peripheral blood or bone marrow by a functional test after the blood or bone marrow has been filtered by a special filtration method. The cells are in particular circulating tumor cells (CTCs), mesenchymal stem cells from peripheral blood or bacteria from blood or other body fluids, and disseminated tumor cells from bone marrow (DTCs).

### BACKGROUND

The appearance of CTCs in peripheral blood is an indication of a possible spread of cells of a solid tumor at a very early stage at which metastasization can still not be detected using customary imaging investigative methods. Consequently, both the detection and the characterization of CTCs in peripheral blood are promising options for detecting systemic tumor cell spread at a very early stage and for utilizing CTCs as prognosis markers. Consequently, prognoses and continuous observation of systemic therapies could be discussed and/or carried out. Furthermore, the characterization and evaluation of CTCs as a diagnostic instrument could be utilized in order to select a suitable treatment for solid tumors.

For the early detection, diagnosis and therapy control of cancer, the detection of circulating tumor cells (CTCs) in the blood has an ever increasing importance. In this connection, on account of the small number of CTCs, which can be in the range of only 3-5 in one milliliter of blood, and on account of the large background of leukocytes (6-10×106 per milliliter), a method has to be chosen which is able to concentrate CTCs as selectively as possible or else is able to isolate them against a large excess of other blood cells. Of use in this connection are e.g. physical methods such as filtration, which permits a size selection of the cells by means of appropriate pore sizes, or other methods, which permit the concentration of CTCs in a blood sample e.g. via elective antibody binding.

No method based on size selection has hitherto been fully automated. Manual operating steps are always necessary between the concentration step and the actual selective immunochemical detection reactions of the CTCs.

Besides flow-cytometric methods (e.g. the FACS method, fluorescence activated cell sorting) and filtration methods, the only available commercial system approved for routine in-vitro diagnostics hitherto is that from Veridex (CellSearch). In a starting volume of a few ml of blood, the method used permits the detection of CTCs if their number to be detected is greater than or equal to 3 per milliliter. The method used for concentration utilizes the specific binding of antibodies coupled to magnetic beads onto the CTCs. The specific detection of the concentrated CTCs takes place visually with the help of fluorescence marked antibodies.

Physical methods are also known which concentrate the CTCs via a size selection and/or deplete and gently “fix” the leukocytes in the blood. In this connection, filters with defined pore sizes are used which permit the permeation of leukocytes and other blood constituents, but are intended to prevent CTCs from slipping through. For the subsequent processing, which consists of a series of washing and selective staining steps, the manual transfer of the filter to a staining station is the customary procedure.

### SUMMARY

By contrast, at least one embodiment of the present invention permits a reliable, cost-effective automatable method for detecting (live) cells in a sample, in particular tumor cells in a blood sample.

At least one embodiment of the invention relates to a method and/or an array.

In a filtration operation in the sense of at least one embodiment of the invention, a suspension is filtered through a filter, e.g. a filter membrane. Here, permeate is pressed through the filter and retentate is retained on the filter surface (and also in the pores and cavities of the filter). During the filtration operation there is therefore a prevailing direction of flow of the permeate through the filter, meaning that it is possible to speak of a region upstream of the filter, in which the retentate is retained (which comprises the cells as an essential constituent), and a region downstream in which the permeate is squeezed and can e.g. be collected there. Irrespective of this prevailing direction of flow, in exceptional cases, the direction of flow can also be reversed, i.e. during a back-flushing the filter.

In order to squeeze the permeate through the filter, a pressure difference can be generated, in which case a higher pressure is then present upstream of the filter than downstream. This can be achieved by applying a superatmospheric pressure upstream of the filter, applying a subatmospheric pressure downstream or a combination of the two. In order to stop the permeate flow through the filter (to reduce to zero), a pressure difference of zero can be established. This is independent of the orientation of the filter in the space. For the special case where the direction of flow at the filter proceeds vertically or a vertical component (thus in the direction of or counter to the force of gravity) proceeds, it must also be taken into consideration that the water column on the filter contributes to the pressure difference.

For some applications of the method according to at least one embodiment of the invention, it is preferred that the direction of flow of the filtration at the filter proceeds essentially in the direction of the gravitational force. As a result, retained retentate comes to rest on the surface of the filter, which e.g. permits simple further processing of the retentate (cells).

For certain applications, it may be preferred not to perform filtration in the direction of the gravitational force, but contrary to the gravitational force, e.g. if the retentate floats, or in order to collect cells after the filtration from the underside of the filter in a collecting vessel.

At least one embodiment of the invention generally relates to a method for detecting cells in a liquid sample, comprising:
a) filtering the liquid sample through a porous membrane that is suitable for retaining cells to be detected, such that cells to be detected are retained on at least part of the surface of the membrane, and at least some of the sample liquid passes the membrane, as permeate,
b) applying a first process liquid containing a first agent for marking the cells to be detected,
c) incubating the first process liquid on the membrane for a predetermined time period, wherein the cells to be detected are marked, and
d) detecting the marked cells to be detected on the surface of the membrane.

### DETAILED DESCRIPTION OF THE EXAMPLE EMBODIMENTS

At least one embodiment of the invention generally relates to a method for detecting cells in a liquid sample, comprising:
a) filtering the liquid sample through a porous membrane that is suitable for retaining cells to be detected, such that cells to be detected are retained on at least part of the surface of the membrane, and at least some of the sample liquid passes the membrane, as permeate,
b) applying a first process liquid containing a first agent for marking the cells to be detected,
c) incubating the first process liquid on the membrane for a predetermined time period, wherein the cells to be detected are marked, and
d) detecting the marked cells to be detected on the surface of the membrane.

The liquid sample can be e.g. a blood sample, urine sample or plasma sample.

According to one embodiment of the invention, step b) is realized by adding the process liquid directly to the liquid sample.

According to one embodiment of the invention, the first process liquid is removed from the marked cells to be detected, e.g. by squeezing through the membrane, prior to detection.

Suitable agents for marking include markers which can stain cells nonspecifically or specifically. Nonspecific markers can be e.g. dyes which stain proteins, nucleic acids or other cell constituents. Specific markers can be e.g. antibodies, probe oligonucleotides, peptides or other molecules which bind specifically onto proteins, nucleic acid sequences or other cell-specific structures. The marker can be marked directly with a detectable marking, e.g. chromogenic dyes, fluorescent dyes, isotope marking or the like. Alternatively, the marker can also be detected via a secondary detection reagent (e.g. secondary antibody or enzyme-substrate system).

In this connection, optionally at least one further process liquid can be applied, e.g. before step b) or after step d).

The at least one further process liquid preferably contains agents which are selected from:

agents for washing, agents for fixing biological structures, agents for preventing nonspecific marking events or agents for marking the cells to be detected with a further marker, where the further marker differs from the first agent for marking.

Appropriate washing buffers are known as agents for washing. They can also comprise agents for permeabilizing cell membranes, e.g. surfactants, saponin and the like.

Agents known for fixing biological structures are corresponding fixing solutions which comprise fixatives, e.g. formalin, glutaraldehyde and the like.

Agents known for preventing nonspecific marking events are corresponding blocking buffers. If e.g. an antibody is used as marker, it is known to saturate nonspecific bindings of the antibody by a blocking buffer which contains nonspecific immunoglobulins of the same species from which the marker antibody stems.

The further process liquid preferably comprises agents which are suitable for washing or fixing the cells or for preventing nonspecific marking events.

According to a preferred embodiment of the invention, in the method, the cells on the membrane are stained with a dye by incubation with a corresponding process liquid after the filtration. For this, dyes can be selected which stain cells or cell constituents and are known from cytology and histology. These can be living or dead dyes, dyes which stain specifically cell cores or other organelles or which specifically stain certain cell components, e.g. nucleic acids or proteins. Known cell dyes are e.g. trypan blue, DAPI and the like.

The cells to be detected can also be analyzed microscopically in unstained or stained form on the membrane.

According to a preferred embodiment of the invention, during step c), a superatmospheric pressure is applied on the side of the membrane which faces away from the side on which the cells to be detected have been retained in step a), the superatmospheric pressure being chosen such that flow of process liquid through the membrane is prevented, the superatmospheric pressure being 50 mbar, 20 mbar, 10 mbar or 5 mbar. The superatmospheric pressure is preferably 3-10 mbar. A slight superatmospheric pressure prevents the process liquid seeping through the membrane.

The superatmospheric pressure is preferably chosen such that, for a direction of flow downwards (in the direction of the gravitational force), it is greater than or equal to the pressure of the water column above the filter. The following applies here: 1 cm water column corresponds to ca. 1 mbar. The water column corresponds to the fill level of the suspension above the filter in the case of an essentially horizontal arrangement of the filter, where filtration takes place from top to bottom.

According to a preferred embodiment of the invention, during step d), a subatmospheric pressure is applied on the side of the membrane which faces away from the side on which the cells to be detected have come to rest in step a), the subatmospheric pressure being selected such that the process liquid is absorbed through the membrane.

According to a preferred embodiment of the invention, the cell to be detected is a tumor cell. The method according to the invention is particularly well suited for detecting CTCs.

According to a preferred embodiment of the invention, in step c), the process liquid and the incubation conditions are chosen such that survival of the cells to be detected over the predetermined time period is permitted. Consequently, it is possible to carry out further functional tests on the cells. Buffers with physiological salt concentrations can be chosen here as process liquid. The incubation can take place under corresponding temperature and humidity conditions.

Furthermore, substances released by the cells to be detected can be detected.

Furthermore, at least one embodiment of the invention relates to an array for carrying out steps a) to d) of the method according to at least one embodiment of the invention, comprising:
a) a porous membrane which is suitable for retaining cells to be detected,
b) a device for applying a liquid to the membrane,
c) a device for squeezing the liquid through the membrane,
d) a device for establishing a pressure difference in front of and behind the membrane,
e) a device for controlling the time progression, which can control the application of the liquid to the membrane, the residence of the liquid on the membrane for a predetermined time period and the squeezing of the liquid through the membrane,
where the device for establishing the pressure difference permit a superatmospheric pressure to be generated on the back of the membrane in order to permit the residence of the liquid on the membrane for a predetermined time period.

The device for applying a liquid to the membrane can be configured e.g. as a feed line or as a pipetting device.

The device for squeezing the liquid through the membrane can include at least one device with which a pressure difference is generated, in which case the pressure upstream of the filter is then higher than that downstream.

The device for controlling the time progression can be envisaged as a programmable controlling of the array in which the predetermined time period can be adjusted.

According to a preferred embodiment of the invention, a superatmospheric pressure is applied on the side of the membrane facing away from the side on which the detectable cells have been retained, the superatmospheric pressure being chosen such that flow of process liquid through the membrane is prevented, the superatmospheric pressure being 50 mbar, 20 mbar, 10 mbar or 5 mbar. The superatmospheric pressure is preferably 3-10 mbar. A slight superatmospheric pressure prevents the process liquid seeping through the membrane.

Preferably, the superatmospheric pressure is selected such that in the case of a direction of flow downwards (in the direction of the gravitational force) it is greater than or equal to the pressure of the water column above the filter. The following applies here: 1 cm water column corresponds to ca. 1 mbar. The water column corresponds to the fill level of the suspension over the filter in an essentially horizontal arrangement of the filter, filtration being performed from top to bottom.

The membrane can be clamped in a corresponding holder, such that corresponding subatmospheric or superatmospheric pressures can be applied.

Preferably, the membrane is arranged on a microscope slide so that after carrying out steps a) to d) of the method according to at least one embodiment of the invention, the microscope slide with the membrane for detecting the cells can be inspected, e.g. by means of investigation using a microscope.

A microscope slide is a transparent plate measuring ca. 26×76 mm (ISO 8255-2) and with a thickness of ca. 0.1 to 1.5 mm.

The microscope slide preferably has openings so that the liquids can be removed by suction without problem. These may be several small openings, e.g. pores or bores. Alternatively, they may also be one or more larger cutouts which can be covered by the membrane.

The membrane has e.g. a pore size of 0.1 to 200 μm. Consequently, cells can be retained whereas cell fragments, thrombocytes and smaller solid constituents of the sample pass through the filter (the membrane).

According to a preferred embodiment of the invention, the membrane has a pore size of 2 to 50 μm, more preferably 5 to 20 μm, even more preferably 5 to 10 μm. Pore sizes in the size ranges 2 to 50 μm, 5 to 20 μm or in particular 5 to 10 μm offer the advantage that the cells are retained thereby, but partly remain stuck in the pores and thus adhere particularly well to the membrane and are available for further analyses.

According to one embodiment of the invention, an antigen from the following list 1 or list 2 is detected with the first marker.

According to a preferred embodiment of the invention, when using a blood sample, prior to the filtration, an erythrocyte lysis (e.g. by hopotonic lysis) is carried out in order to remove troublesome erythrocytes.

Furthermore, after the filtration, the cells can also be taken up again in cell culture medium and be cultivated for further investigations. Thus, e.g. detected tumor cells can be cultivated and further investigated in order to test the response to certain drugs (e.g. cytostatics).

List 1: preferred cell-specific antigens:
alpha-l-fetoprotein (AFP) in liver cell carcinoma and gonadal and extragonadal germ cell tumors
Bence-Jones protein in multiple myeloma
Beta-HCG (beta subunit of human choriongonadotropin) in germ cell tumors of the ovary and non seminomatous tumors of the testicle
CA 15-3 in breast cancer (mammary carcinoma) or ovarian cancer (ovarian carcinoma)
CA 19-9 and CA 50 in pancreatic cancer (pancreatic carcinoma)
CA-125 in ovarian cancer (ovarian carcinoma)
Calcitonin (human calcitonin, hCT), in medullary thyroid carcinoma
Carcinoembryonic antigen (CEA) in stomach cancer, pancreatic carcinoma and adenocarcinoma of the lung
Cytokeratin-21 fragment (CYFRA 21-1) and Serpin B4 (SCC) for all variants of lung cancer (bronchial carcinoma)
HER-2/neu
HPV antibodies and HPV antigens
Homovanillinic acid in neuroblastoma
5-hydroxyindoleacetic acid in carcinoid
catecholamines, vanillylmandelic acid in pheochromocytoma
lactate dehydrogenase (LDH) in germ cell tumors
lactate dehydrogenase isoenzyme 1 (LDH-1) in germ cell tumors; a routine determination, is still not recommended however in current guidelines
MAGE antigens metanephrines in pheochromocytoma
MUC1 in non-small-cell bronchial carcinoma (NSCLC) or in mammary carcinoma
NSE in small-cell bronchial carcinoma (SCLC), neuroblastoma, and seminomatous germ cell tumors
placental alkaline phosphatase (PLAP) in seminomatous germ cell tumors
PSA in prostate cancer (prostate carcinoma)
thyreoglobulin (Tg) in any concentration in papillary or follicular thyroid carcinoma
thymidine kinase
cytokeratins, e.g. cytokeratin 8, 18, 19

List 2: additional cell-specific antigens
β2-microglobulin (β2-M),
CA 54-9,
CA 72-4,
CA 195,
Cancer Associated Serum Antigen (CASA),
C-peptide,
cytokeratin,
gastrin,
glucagon,
glucose-6-phosphate isomerase (GPI),
insulin,
neopterin,
nuclear matrix protein 22 (NMP 22),
ostase,
P53 autoantibodies,
paraproteins,
prolactin (PRL),
protein S-100,
serpin B4 (SCC),
pregnancy-specific β1-glycoprotein (SP-1),
tumor-associated glycoprotein 12 (TAG 12),
thymidine kinase (TK),
tissue polypeptide antigen (TPA),
tissue polypeptide specific antigen (TPS),
tumor M2-PK,
vasoactive intestinal polypeptide (VIP),
transketolase-like 1 protein (TKTL1)

The antigens specified in list 1 and 2 are exemplary targets for specific markers (e.g. antibodies), via which, according to the method of an embodiment of the invention, cells, in particular tumor cells, can be detected.

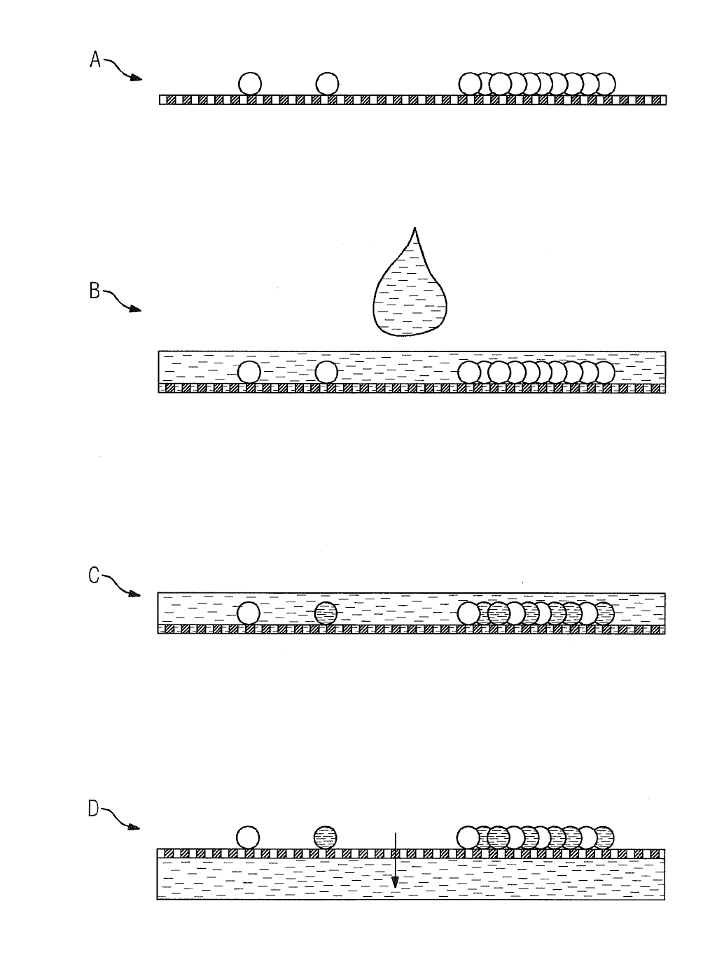

The method according to an embodiment of the invention is described below by way of example. Attached FIG. 1 shows diagrammatically steps a) to d) of the method according to an embodiment of the invention.

Step A shows the filtering of the liquid sample through a porous membrane in such a way that cells to be detected are retained and/or come to rest on the surface of the membrane,

Step B shows the application of a first process liquid which contains a first agent for marking the cells to be detected,

Step C shows the incubating of the process liquid on the membrane for a predetermined time period, where the cells to be detected are marked (shown by hatching of the marked cells), and

Step D shows the removal of the process liquid, e.g. by suction.

The solids (cell, particle, tissue) to be separated from the surrounding medium (sample liquid) are separated from the medium as a result of the fact that they remain on the surface of a filter membrane which is impermeable for the solids to be separated, but is permeable for the surrounding medium and also for solids contaminating the solid to be separated. The devices provided for this are those which allow the flow rate of the gas or of the liquid through the surface permeable therefor to be determined and modified in a targeted manner. Preferably, in medical diagnostics, the solids are cellular components and the surrounding medium is full blood/serum/plasma, which can also contain particles for which, however, the surface is permeable on account of its properties (e.g. leukocytes).

As a result of an automated procedure, as well as the sample, all of the reagents necessary for the isolation/concentration and the detection are applied to the surface of the membrane in a targeted manner, for example by means of a pipetting robot. The sample and the reagents can remain in a controlled manner on the surface for incubation purposes and/or be removed by the surface permeable for them. This is achieved through the use of a variety of sensors and actuators which permit passage, regulated according to the requirements, of the substances through the permeation layer (permeable surface) of the membrane. Here, the regulation can take place for example according to the flow rate or else also, in the case of particularly sensitive cellular components, by means of a pressure difference (to the ambient pressure) placed on the system.

All of the steps, from the application of the medium to the semipermeable surface to the selective marking, are preferably carried out automatically and monitored in a liquid handling robot. In this connection, a simultaneous, parallel processing of a large number of samples to be investigated can be achieved.

As a result of operating the sequential isolation/concentration protocol and the selective detection reactions in one instrument unit, without the hitherto customary manual interim steps and sample transfer, a less error-susceptible analysis and a more rapid runtime are achieved. Since, in the method proposed here, also the incubation of all process liquids and/or reagents necessary for the detection is regulated by way of targeted application to the semipermeable surface and the controlled flow through this surface, the reagent volumes used can be significantly reduced, which represents a considerable cost advantage for the often high-cost immunochemical reagents. Both in the case of manual immunochemical stains and also in the case of the known “autostainers”, the incubation volume is significantly larger since not only does the surface to be treated have to be wetted, but a very much larger area is flushed and/or the entire incubation vessel has to be filled with an adequate volume.

To carry out the concentration/washing and staining steps, it is necessary:
(a) the liquid volume or the fill level above the semipermeable surface
(b) the rate of permeation of the liquid through the semipermeable surface
(c) to know the pressure difference required for the permeation between the surrounding area and the apparatus and to use it for controlling the permeation rate and for determining the currently analyzed sample volume.

The sensorics and actorics necessary therefor can either be a constituent of the permeation apparatus or of the pipetting robot, or else be divided, which necessitates communication between the components.

For the individual controlling of each individual sample/channel, at least one pressure sensor and one valve is required with which the pressure difference relative to the surrounding area on the system (i.e. the superatmospheric pressure or subatmospheric pressure) can be regulated. The particular pressure difference is established for example by virtue of the connection with a storage vessel in which either a subatmospheric pressure or a superatmospheric pressure prevails relative to the surroundings.

Monitoring the through-flow of liquid can advantageously take place by means of a liquid handling robot which adopts the following steps for this purpose:
(a) channel-specific fill level measurement (e.g. capacitive) for determining the liquid volume above the semipermeable membrane for the overflow control and controlling the time of the after-pipetting,
(b) converting the captured measurement data into data which can be read by external software and used for further processing,
(c) depending on the particular fill level, the additional pipetting of a further liquid aliquot.

The rate at which the permeation of the medium through the semipermeable surface takes place is advantageously controlled either via the flow rate or the pressure difference required for the permeation. For this purpose, in the case of sensitive components to be isolated, an extremely low pressure difference e.g. of up to 7 mbar is applied, although this can also increase to up to 50 mbar. The continual measurement of the pressure difference, however, also serves for establishing whether there is still sample material above the semipermeable membrane, and can thus serve as a signal for the completed permeation, or else be used as an analysis switch-off signal if a blockage of the semipermeable surface results during the course of the assay.

As a result of the portioned addition of the sample, e.g. by way of a liquid-handling robot, and occasional mixing of the sample, it is possible to largely avoid sedimentation of the cells and associated cell loss.

The automated steps for the concentration and the immunochemical detection or the molecular biological characterization of CTCs in a blood sample are described by way of example.
(i) Manual processing steps:
introduction of the blood samples to be investigated into the instrument
introduction of the required prepared reagents
introduction of the other consumables (e.g. pipette tips)
after analysis: removal of the waste

(ii) Course of the automated CTC concentration and immunochemical CTC detection:
mixing of the blood samples
removal of the blood sample and addition to a dilution/digestion/fixing buffer
incubation of blood sample and buffer
optional conditioning of the membrane by a series of washing and incubation steps
pipetting of the treated blood sample into the arrangement such that the membrane is covered.
concentration, controlled by fill level and pressure difference, of the CTCs on the membrane (e.g. as a result of fill level sensorics in the pipette tip, pressure measurement and control of the pressure difference by means of a valve to the sub/superatmospheric pressure storage vessel)
switching off of the permeation process for the permanent addition of the membrane
mild fixing and washing of the cells on the semipermeable membrane
immunochemical staining by incubation of the cells on the membrane with the necessary process liquids/reagents
assay progress controlled by fill level and pressure difference (fill level sensorics in the pipette tip, pressure measurement and control of the pressure difference by means of a valve to the sub/superatmospheric pressure storage vessel)
switching off of the permeation process for the permanent addition of membrane

Optionally connected downstream: (c) “molecular biological characterization” by FISH (fluorescence in-situ hybridization)
molecular biological staining by means of heated incubation on the membrane with the necessary reagents
prevention of evaporation in the event of extended incubation by placing on a cover glass which is removed again after incubation.

(iv) Final steps
pipetting of a suitable mounting medium, which may also be solidifying, onto the semipermeable membrane
placing on a cover glass step (iv) can optionally take place manually again.

During the so-called immunostaining, body cells or cell associations (tissue sections) are (mostly sequentially) incubated e.g. with antibody solutions, label reagents, washing buffers and many types of process liquids. Typically, the stained specimens are analyzed on a microscope slide using a microscope.

The fluidic process steps required for the staining are very diverse and complex. Some of the reagents used are very expensive. Automated processing on a microscope slide hardly permits cost-saving processing with little volume. The reagents are mostly flushed away over the microscope slide which, besides a high consumption of reagents, may in some instances also be associated with a partial loss of specimens.

According to at least one embodiment of the invention, the reagents are not finished away over a surface, but flushed through a membrane, which saves reagents, avoids the loss of specimens and permits a generally better process control.

1. A method for detecting cells in a liquid sample, comprising:
a) filtering the liquid sample through a porous membrane, suitable for retaining cells to be detected, such that cells to be detected come to rest on at least part of the surface of the porous membrane, and at least some of the sample liquid passes the porous membrane;
b) applying a first process liquid containing a first agent for marking the cells to be detected with a first marker;
c) incubating the process liquid on the membrane for a time period, wherein the cells to be detected are marked; and
d) detecting the marked cells to be detected on the surface of the membrane.

2. The method of claim 1, wherein before step b) or after step c), at least one further process liquid is applied.

3. The method of claim 1, wherein the at least one further process liquid contains agents which are selected from agents for washing, agents for fixing biological structures, agents for preventing nonspecific marking events or agents for marking the cells to be detected with a further marker, and wherein the further marker differs from the first marker.

4. The method of claim 1, wherein the second process liquid contains agents which are suitable for the washing or fixing of the cells or for the prevention of nonspecific marking events.

5. The method of claim 1, wherein during step c), a superatmospheric pressure is present on the side of the membrane which faces away from the side on which the cells to be detected come to rest in step a), the superatmospheric pressure being selected such that flow of process liquid through the membrane is prevented.

6. The method of claim 1, wherein during step d), a subatmospheric pressure is present on the side of the membrane which faces away from the side on which the cells to be detected come to rest in step a), the subatmospheric pressure being selected such that the process liquid is absorbed through the membrane.

7. The method of claim 1, wherein the cells to be detected are tumor cells.

8. The method of claim 1, wherein in step c), the process liquid and the incubation conditions are chosen to permit survival of the cells to be detected over the time period.

9. The method of claim 1, wherein substances released from the cells to be detected are detected.

10. An array, comprising:
a) a porous membrane, suitable for retaining cells to be detected;
b) a device for applying a liquid to the membrane;
c) a device for squeezing the liquid through the membrane;
d) a device for establishing a pressure difference in front of and behind the membrane; and
e) a device for controlling a time progression which can control the application of the liquid to the membrane,
residence of the liquid on the membrane for a time period and the squeezing of the liquid through the membrane, wherein agents for establishing the pressure difference permit a superatmospheric pressure of ≦50 mbar to be generated on the back of the membrane in order to permit the residence of the liquid on the membrane for the time period.

11. The array of claim 10, wherein the membrane is arranged on a microscope slide.

12. The array of claim 10, further comprising:
a waste container for collecting liquids squeezed through the membrane.

13. The method of claim 2, wherein the at least one further process liquid contains agents which are selected from agents for washing, agents for fixing biological structures, agents for preventing nonspecific marking events or agents for marking the cells to be detected with a further marker, and wherein the further marker differs from the first marker.

14. The array of claim 11, further comprising:
a waste container for collecting liquids squeezed through the membrane.
